(19) United States
(12) Patent Application Publication
(10) Pub. No.: US 2020/0407304 A1
(43) Pub. Date: Dec. 31, 2020

(54) PROCESS AND INTERMEDIATE FOR THE MANUFACTURE OF DIFLUOROACETYL CHLORIDE

(71) Applicant: SOLVAY SA, Brussels (BE)

(72) Inventors: Etienne SCHMITT, Hannover (DE); Janis JAUNZEMS, Hannover (DE)

(21) Appl. No.: 16/643,962

(22) PCT Filed: Sep. 4, 2018
(86) PCT No.: PCT/EP2018/073670

§ 371 Date: Mar. 3, 2020

(30) Foreign Application Priority Data
Sep. 4, 2017 (EP) 17189146.8

(51) Int. Cl. C07C 49/16

(52) U.S. Cl. CPC C07C 49/16

(57) ABSTRACT

The present invention concerns a process and intermediates for the manufacture of difluoro acetyl chloride. The invention further concerns a process for the manufacture of an agrochemically or pharmaceutically active compound, which comprises the process and intermediate for the manufacture of difluoro acetyl chloride for the manufacture of difluoro acetyl chloride or its intermediate.

This application claims priority to European application No. 17189146.8, the whole content of this application being incorporated herein by reference for all purposes.

The present invention concerns a process and intermediates for the manufacture of difluoroacetyl chloride. The invention further concerns a process for the manufacture of an agrochemically or pharmaceutically active compound, which comprises the process and intermediates for the manufacture of difluoroacetyl chloride for the manufacture of difluoroacetyl chloride or its intermediate.

It is known that difluoroacetyl chloride is useful as an intermediate for pharmaceutically and agrochemically active compounds and as a reaction reagent, particularly a reagent for the introduction of a difluoromethyl group or difluoroacetyl group into an organic compound. Difluoro acetic acid fluoride, which is commonly manufactured from 1-alkoxy-1,1,2,2-tetrafluoroethane, often is contaminated with HF, which can, even in traces, cause side reactions when used in downstream processes, and is thus, also due to a difference in reactivity, in many cases no suitable replacement for difluoroacetyl chloride.

EP2554534A1 discloses the manufacture of difluoroacetyl chloride (DFAC) from 1-alkoxy-1,1,2,2-tetrafluoroethane wherein 1-alkoxy-1,1,2,2-tetrafluoroethane is brought into contact with CaCl₂ at reaction enabling temperatures, which are reported to be at least 50° C., and in practice are 200° C. and higher.

It was now found surprisingly that 1-alkoxy-1,1,2,2-tetrafluoroethane can be converted into DFAC by reacting a 1-alkoxy-1,1,2,2-tetrafluoroethane with at least one chloride compound selected from the group consisting of SiCl₄, TiCl₄, ZnCl₂ and AlCl₃. This reaction has been found to work well at low temperatures, for example room temperature, which, among other advantages, is less costly due to energy saving and lower complexity of equipment, has a lower risk of unwanted side products such as HF, and includes the option to work the reaction in a liquid or solid/liquid medium without being bound to be working in the gas phase. It has further been found that the reaction can be controlled such as to proceed step-wise via the intermediary of formula (III) as defined below, which improves process control, side-reactions, workup and yield.

The new compounds of formula (III) according to the present invention have been found to be useful intermediates for the manufacture of DFAC.

In the present invention, designations in singular are in intended to include the plural; for example, “a solvent” is intended to denote also “more than one solvent” or “a plurality of solvents”.

All aspects and embodiments of the present invention are combinable.

In the context of the present invention, the term “comprising” is intended to include the meaning of “consisting of”.

When a bond, in particular a double bond, is depicted in a particular geometry, this is intended to also denote other isomeric forms as well as mixtures thereof.

The invention concerns a process for the manufacture of a compound of formula (I) or at least one compound of formula (III), or a mixture of the compound of formula (I) and at least one compound of formula (III), wherein a compound of formula (II) is reacted with at least one chloride compound selected from the group consisting of SiCl₄, TiCl₄, ZnCl₂ and AlCl₃

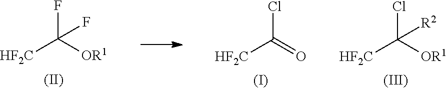

wherein R¹ is selected from the group consisting of C₁-C₁₂-alkyl groups, C₃-C₁₀-cycloalkyl groups, aryl and heteroaryl groups, and wherein R² is F or Cl.

The term “C₃-C₁₀-cycloalkyl” intends to denote mono-, bi- or tricyclic hydrocarbon groups comprising 3 to 10 carbon atoms, in particular 3 to 6 carbon atoms. Examples of monocyclic groups include cyclopropyl, cyclobutyl, cyclopentyl, cyclohexyl, cycloheptyl or cyclooctyl. Examples of bicyclic groups include bicyclo[2.2.1]heptyl, bicyclo[3.1.1]heptyl, bicyclo[2.2.2]octyl and bicyclo[3.2.1]octyl. Examples of tricyclic groups are adamantyl and homoadamantyl. The “C₃-C₁₀-cycloalkyl” are optionally substituted. The substituted C₃-C₁₀-cycloalkyl groups can be selected, for example, from cyclobutyl, cyclopentyl, 2-methylcyclopentyl, 3-methylcyclopentyl, 2-ethylcyclopentyl, 3-ethylcyclopentyl, cyclohexyl, 2-methylcyclohexyl, 3-methylcyclohexyl, 4-methylcyclohexyl, 2-ethylcyclohexyl, 3-ethylcyclohexyl, 4-ethylcyclohexyl, cycloheptyl, 2-methylcycloheptyl, 3-methylcycloheptyl, 3-methylcycloheptyl and 4-methylcycloheptyl.

The term “aryl group” intends to denote C₅-C₁₈ monocyclic and polycyclic aromatic hydrocarbons with 5 to 18 carbon atoms in the cyclic system. Specifically, this definition comprises, for example, the meanings cyclopentadienyl, phenyl, cycloheptatrienyl, cyclooctatetraenyl, naphthyl and anthracenyl. The aryl group are optionally substituted. The substituted aryl groups can be selected, for example, from 2-methylphenyl, 3-methylphenyl, 4-methylphenyl, 2,3-dimethylphenyl, 2,4-dimethylphenyl, 2,5-dimethylphenyl, 2,6-dimethylphenyl, 3,4-dimethylphenyl, 3,5-dimethylphenyl, 3,6-dimethylphenyl, 2-methoxyphenyl, 3-methoxyphenyl and 4-methoxyphenyl.

The term “heteroaryl group” intends to denote C₅-C₁₈ monocyclic and polycyclic aromatic hydrocarbons with 5 to 18 carbon atoms in the cyclic system, wherein one or more methine (—C═) and/or vinylene (—CH═CH—) groups are replaced by trivalent or divalent heteroatoms, in particular nitrogen, oxygen and/or sulphur, respectively, in such a way as to maintain the continuous π-electron system characteristic of aromatic systems. Specifically, this definition comprises, for example, the meanings 2-furyl, 3-furyl, 2-thienyl, 3-thienyl, 2-pyrrolyl, 3-pyrrolyl, 3-isoxazolyl, 4-isoxazolyl, 5-isoxazolyl, 3-isothiazolyl, 4-isothiazolyl, 5-isothiazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, 1,2,4-oxadiazol-3-yl, 1,2,4-oxadiazol-5-yl, 1,2,4-thiadiazol-3-yl, 1,2,4-thiadiazol-5-yl, 1,2,4-triazol-3-yl, 1,3,4-oxadiazol-2-yl, 1,3,4-thiadiazol-2-yl and 1,3,4-triazol-2-yl; 1-pyrrolyl, 1-pyrazolyl, 1,2,4-triazol-1-yl, 1-imidazolyl, 1,2,3-triazol-1-yl, 1,3,4-triazol-1-yl; 3-pyridazinyl, 4-pyridazinyl, 2-pyrimidinyl, 4-pyrimidinyl, 5-pyrimidinyl, 2-pyrazinyl, 1,3,5-triazin-2-yl and 1,2,4-triazin-3-yl. Generally, a heteroaryl group can optionally be substituted.

In the context of the present invention, the term “C₁-C₁₂-alkyl groups” is intended to denote straight or branched alkyl groups having one to twelve carbon atoms. The group comprises, for example, n-nonyl and its isomers, n-decyl and its isomers, n-undecyl and its isomers and n-dodecyl and its isomers, methyl, ethyl, n-propyl, isopropyl, n-, iso-, sec- and t-butyl, n-pentyl and its isomers, n-hexyl and its isomers, 1,3-dimethylbutyl, 3,3-dimethylbutyl, n-heptyl and its isomers and n-octyl and its isomers. Often, the C₁ to C₄ alkyl groups are the preferred groups of the C₁-C₁₂ alkyl group. The term “C₁-C₄-alkyl group” is intended to denote straight or branched alkyl groups having one to four carbon atoms. This group comprises methyl, ethyl, n-propyl, isopropyl, n-, iso-, sec- and t-butyl. The compound of formula (II) can be selected, for example, from the group consisting of 1-methoxy-1,1,2,2-tetrafluoroethane, 1-ethoxy-1,1,2,2-tetrafluoroethane, 1-(n-propoxy)-1,1,2,2-tetrafluoroethane, 1-isopropoxy-1,1,2,2-tetrafluoroethane, and 1-(n-butoxy)-1,1,2,2-tetrafluoroethane; 1-methoxy-1,1,2,2-tetrafluoroethane and 1-ethoxy-1,1,2,2-tetrafluoroethane are the most preferred compounds (II). According to the present invention, ethyl and methyl are the most preferred group R¹ in the compounds (II) and (III). According to a preferred aspect of the present invention, R¹ is a C₁-C₁₂-alkyl group, more preferably a C₁-C₄-alkyl group, and most preferably methyl or ethyl.

In the process wherein a compound of formula (II) is reacted with at least one chloride compound selected from the group consisting of SiCl₄, TiCl₄, ZnCl₂ and AlCl₃, a compound of formula (I) or at least one compound of formula (III), or a mixture of the compound of formula (I) and at least one compound of formula (III) is obtained. The reaction conditions, such the at least one chloride compound, temperature, reaction time, compound of formula (II), absence or presence of a solvent, will determine whether a compound of formula (I) or at least one compound of formula (III), or a mixture of the compound of formula (I) and at least one compound of formula (III) is obtained. Without wishing to be bound to a theory, when a compound of formula (II) is reacted with at least one chloride compound selected from the group consisting of SiCl₄, TiCl₄, ZnCl₂ and AlCl₃ and a compound of formula (I) is obtained, the at least one compound of formula (III) might be formed as an intermediate in the reaction, even when the at least one compound of formula (III) is not detected in the reaction mixture or reaction product. Investigations have shown that the process for the manufacture of (I) from (II) directly or at least one compound of formula (III) according to the present invention does not involve intermediary formation of difluoroacetyl fluoride.

The invention concerns also a process for the manufacture of a compound of formula (I), wherein at least one compound of formula (III) is reacted with at least one compound selected from the group consisting of chloride compounds SiCl₄, TiCl₄, ZnCl₂, phosphorus pentachloride, thionyl chloride, SbCl₃, SbCl₅, AlCl₃ and metal oxide,

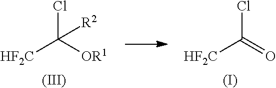

wherein R¹ is selected from the group consisting of C₁-C₁₂-alkyl, C₃-C₁₀-cycloalkyl, aryl and heteroaryl, and each of the foregoing is defined as above, and, and wherein R² is F or Cl. In this reaction, chloride compounds SiCl₄, TiCl₄, ZnCl₂, phosphorus pentachloride, thionyl chloride, SbCl₃, SbCl₅, AlCl₃ and metal oxide serve as catalysts to the reaction of the compound of formula (III) into the compound of formula (I). Suitable metal oxides include aluminium, zirconium or titanium oxides, also denoted as alumina (Al₂O₃), zirconia (ZrO₂) and titania (TiO₂). The metal oxide according to this aspect can contain other atom species than the metal component and oxygen. As such other atom species, a fluorine atom and a chlorine atom are, for example, preferred. For example, the metal oxide catalyst may be partially fluorinated alumina, partially chlorinated alumina, partially fluorinated and chlorinated alumina, partially fluorinated zirconia or partially fluorinated titania. The proportion of the chlorine atom or the fluorine atom in such a metal oxide catalyst is not particularly limited. The metal oxide is preferably subjected to activation treatment prior to the reaction for the manufacture of a compound of formula (I), wherein at least one compound of formula (III) is reacted with at least one compound selected from the group consisting of chloride compounds SiCl₄, TiCl₄, ZnCl₂, AlCl₃ and metal oxide. The activation treatment can be a common method and is not particularly limited. As such activation treatment, it is preferred to sufficiently dehydrate the catalyst in a nitrogen stream at a temperature of from 250° C. to 300° C. and to activate it with dichlorodifluoromethane, chlorodifluoromethane or hydrogen fluoride.

The process for the manufacture of a compound of formula (I), wherein at least one compound of formula (III) is reacted with at least one compound selected from the group consisting of chloride compounds SiCl₄, TiCl₄, ZnCl₂, AlCl₃, phosphorus pentachloride, thionyl chloride, SbCl₃, SbCl₅, and metal oxide, in one aspect further comprises the step wherein the at least one compound of formula (III) is manufactured by reacting a compound of formula (II) with at least one compound selected from the group consisting of chloride compounds SiCl₄, TiCl₄, ZnCl₂ and AlCl₃ as described above. Preferred chloride compounds and residues are defined as before.

In the manufacture of compound (I) and/or at least one compound of formula (III), the amount of chloride compound often is from equal to or more 0.4 to equal to or less than 1.6 molar equivalents, based on the amount of compound (II). This amount, when a mixture of more than one chloride compound is present, denotes the combined amount of chloride compounds present in the reaction. A preferred amount of chloride compounds is from equal to or more than 0.5 to equal to or less than 1 equivalent.

In a preferred aspect, in the for the manufacture of a compound of formula (I) and/or (III), the compound of formula (II) is reacted with at least one chloride compound selected from the group consisting of SiCl₄, TiCl₄, ZnCl₂ and AlCl₃ in combination with at least one metal oxide. The term “metal oxide” is defined as above, wherein Al₂O₃ is preferred. Particularly advantageous is a combination of SiCl₄ and a metal oxide, wherein Al₂O₃ is preferred as metal oxide. In the process for the manufacture of the compound of formula (III) by reacting the compound of formula (II) with at least one chloride compound selected from the group consisting of SiCl₄, TiCl₄, ZnCl₂ and AlCl₃ in combination with at least one metal oxide, wherein Al₂O₃ is preferred, and the combination of Al₂O₃ and SiCl₄ is most preferred, the temperature often is from and including −10° to and including 80° C., preferably from and including 0° to and including 70° C., and more preferably from and including 10° to and including 60° C. When Al₂O₃ is present in the conversion of compound (II) into compound (III), preferably in the presence of SiCl₄, a bed of Al₂O₃ can advantageously be employed. To improve further the turnover of the reaction, more than one bed reactor can be employed, or the reaction product mixture can be looped through one or more reactors until maximum conversion is achieved. If it is desired to isolate (III), it is preferred that the metal oxide, preferably Al₂O₃ is removed prior to isolation of (III). In this respect, using a bed reactor is even more advantageous, as Al₂O₃ is not present in the reaction mixture at the end of the step of converting (II) into (III).

In another preferred aspect, in the for the manufacture of a compound of formula (I) from the compound of formula (III), the compound of formula (III) is reacted with at least one chloride compound selected from the group consisting of SiCl₄, TiCl₄, ZnCl₂, phosphorus pentachloride, thionyl chloride, SbCl₃, SbCl₅ and AlCl₃ in combination with at least one metal oxide. The term “metal oxide” is defined as above, wherein Al₂O₃ is preferred. Particularly advantageous is a combination of SiCl₄ and a metal oxide, wherein Al₂O₃ is preferred as metal oxide. When Al₂O₃ is present in the conversion of compound (III) into compound (I), preferably in the presence of SiCl₄, a bed of Al₂O₃ can advantageously be employed. To improve further the turnover of the reaction, more than one bed reactor can be employed, or the reaction product mixture can be looped through one or more reactors until maximum conversion is achieved.

In one preferred aspect, when the at least one compound of formula (III) is manufactured by reaction of a compound of formula (II), the chloride compound is TiCl₄. In this aspect, the molar equivalent often is from equal to or more than 1 to equal to or less than 1.5 molar equivalents based on the amount of compound (II).

According to the present invention, when the compound of formula (I) is manufactured by reaction of a compound of formula (II), the preferred chloride compound in one aspect is SiCl₄, AlCl₃ or a mixture of SiCl₄ and AlCl₃. A mixture of SiCl₄ and AlCl₃ is particularly preferred. The preferred chloride compound which in one aspect is SiCl₄, AlCl₃ or a mixture of SiCl₄ and AlCl₃, preferably is present in a molar equivalent of from 0.6 to 1.0, based on the amount of compound (II). When a mixture of SiCl₄ and AlCl₃ is present, the ratio of AlCl₃:SiCl₄ is often 1:1, and preferably 1:2.

In the processes for the manufacture of a compound of formula (I), one compound of formula (III) or mixtures thereof, starting from (II) or (III) as described before generally are conducted at a temperature of equal to or more than 0° C. to equal to or less than 90° C., wherein preferably the reaction temperature is from equal to or more than 10° C. to less than 50° C. Temperatures of from equal to or more than 15° C. to equal to or less than 30° C. are most preferred.

According to one aspect of the present invention, the processes for the manufacture of a compound of formula (I), (III) or mixtures thereof, starting from (II) or (III) as described before, generally can be performed in the absence of additional solvents. The absence of solvents is preferred for the manufacture of the compound of formula (I) from the compound of formula (II) when the at least one chloride compound is SiCl₄, AlCl₃ or a mixture of SiCl₄ and AlCl₃. The processes which are conducted in the absence of solvents preferably are conducted at temperatures of equal to or more than 10° C. to less than 50° C., wherein temperatures of from equal to or more than 15° C. to equal to or less than 30° C. are preferred. The term “absence of solvents” intends to denote the absence of added solvents; reagents such as compound of formula (II) or (III), products such as compounds (I) and/or (III) and chloride compounds which are liquid under the reaction conditions are not considered to be solvents.

According to another aspect of the present invention, the processes for the manufacture of a compound of formula (I), (III) or mixtures thereof, starting from (II) or at least one compound of formula (III) as described before, generally can be performed in the presence of at least one additional solvent. The presence of at least one additional solvent is preferred for the manufacture of the compound of formula (III) from the compound of formula (II), and is particularly preferred when the at least one chloride compound is TiCl₄. The processes which are conducted in the presence of at least one additional solvent generally are conducted at temperatures 0° C. to equal to or less than 90° C. The at least one solvent is selected from the group of inert solvents, wherein the term “inert” intends to denote that the at least one solvent is not participating in the reaction. Suitable solvents include hydrocarbons, halogenated hydrocarbons, ethers, carboxylic acids, esters and amides. Specific examples of the reaction solvent are benzene, toluene, chlorobenzene, 1,2,4-trichlorobenzene, xylene, ethylbenzene, dichloromethane, isopropylbenzene, tetrachloroethylene, acetonitrile, sulfolane, Me₂SO₂, Ph₂SO₂, Et₂SO₂, tetralin, dodecane, mesitylene, tetrahydrofuran, methyltetrahydrofuran, CPME (Cyclopentyl methyl ether), 2-methylglutaronitrile, dimethoxyethane, 1,4-dioxane, diethyl ether, acetic acid, ethyl acetate and dimethylformamide. Acetonitrile, dichloromethane and ethyl acetate are preferred solvents. TiCl₄ preferably is used in the presence of a coordinating solvent, such as sulfolane, Me₂SO₂, Ph₂SO₂, Et₂SO₂ or nitriles such as 2-methylglutaronitrile or acetonitrile. It is believed that TiCl₄ forms a complex with the coordinating solvent, as decribed in P. Biagini et al, Dalton Trans. 2004, 2364-2371. The conversion from the compound of formula (II) into (III) can be particularly well controlled when TiCl₄ in the presence of a coordinating solvent, in particular sulfolane, is used. The obtained compound of formula (III) can then be transferred into the compound of formula (I) by employing elevated temperatures, for example from 70 to and including 110° C., preferably from 80 to and including 100° C. The intermediary compound of formula (III) does not need to be isolated before its conversion into the compound of formula (I).

Another object of the present invention is a compound of formula (III)

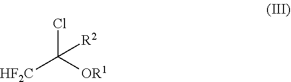

wherein R¹ is selected from the group consisting of C₁-C₁₂-alkyl, C₃-C₁₀-cycloalkyl, aryl and heteroaryl, and wherein R² is F or Cl. Preferably, R¹ is selected from the group consisting of C₁ to C₄ alkyl groups. Most preferably, R¹ is selected from the group consisting of ethyl and methyl. Preferred compounds thus are 1-chloro-1,2,2-trifluoro-1-methoxyethane, 1,1-dichloro-2,2-difluoro-1-methoxyethane, 1,1-dichloro-1-ethoxy-2,2-difluoroethane and 1-chloro-1-ethoxy-1,2,2-trifluoroethane. As described before, the compounds of formula (III) can be obtained by reaction of the compound of formula (II) with at least one compound selected from the group consisting of chloride compounds SiCl₄, TiCl₄, ZnCl₂ and AlCl₃. TiCl₄ is the most preferred chloride compound for the manufacture of the compound of formula (III). The compounds of formula (III) are useful for the manufacture of compounds of formula (I) as described before. Similarly, the compound of formula (III) can also be defined by replacing the CF₂H group by a C₁ to C₄ alkyl group which is substituted with at least one halogen atom, such as the group CF₃, CCl₂Br, CClHBr, CH₂F, CCl₃, CHBr₂ or CHCl₂. The CF₂H group of the starting material of formula (II) is similarly adapted to obtain such a compound (III) with alternative C₁ to C₄ group which is substituted with at least one halogen atom.

Another object of the present invention is a process for the manufacture of a pharmaceutically or agrochemically active compound, which comprises the process for the manufacture of a compound of formula (I) and/or at least one compound of formula (III). In one aspect, the process for the manufacture of a compound of formula (I) and/or at least one compound of formula (III) is comprised a process wherein the compound of formula (I) is reacted with a vinyl ether to obtain a halogenated alkenone. Principles of such a reaction are described in WO2010000871 and WO2004/108647. Halogenated alkenones are useful for the manufacture of heterocycles which are intermediates for the manufacture of agrochemically and pharmaceutically active compounds, as described, for example, in EP0744400 and WO2010037688. In one aspect, the process for the manufacture of a compound of formula (I) and/or at least one compound of formula (III) is comprised in a process wherein the compound of formula (I) is reacted with an acrylic acid derivate. Such reactions are particularly suitable for further manufacturing pyrazoles carboxylic acids, as described in WO05042468, WO03051820, WO16152886, WO2017/064550 and WO2017/129759. All of the foregoing references are incorporated hereby by reference for all purposes. The pyrazole carboxylic acids are particularly useful in the manufacture of carboxamide fungicides, in particular Sedaxane, Fluopyram, Benzovindiflupyr, Bixafen, Fluxapyroxad, Isopyrazam, Penflufen and Penthiopyrad.

In one specific aspect, the invention concerns the process for the manufacture of the compound of formula (VIII) or (IX)

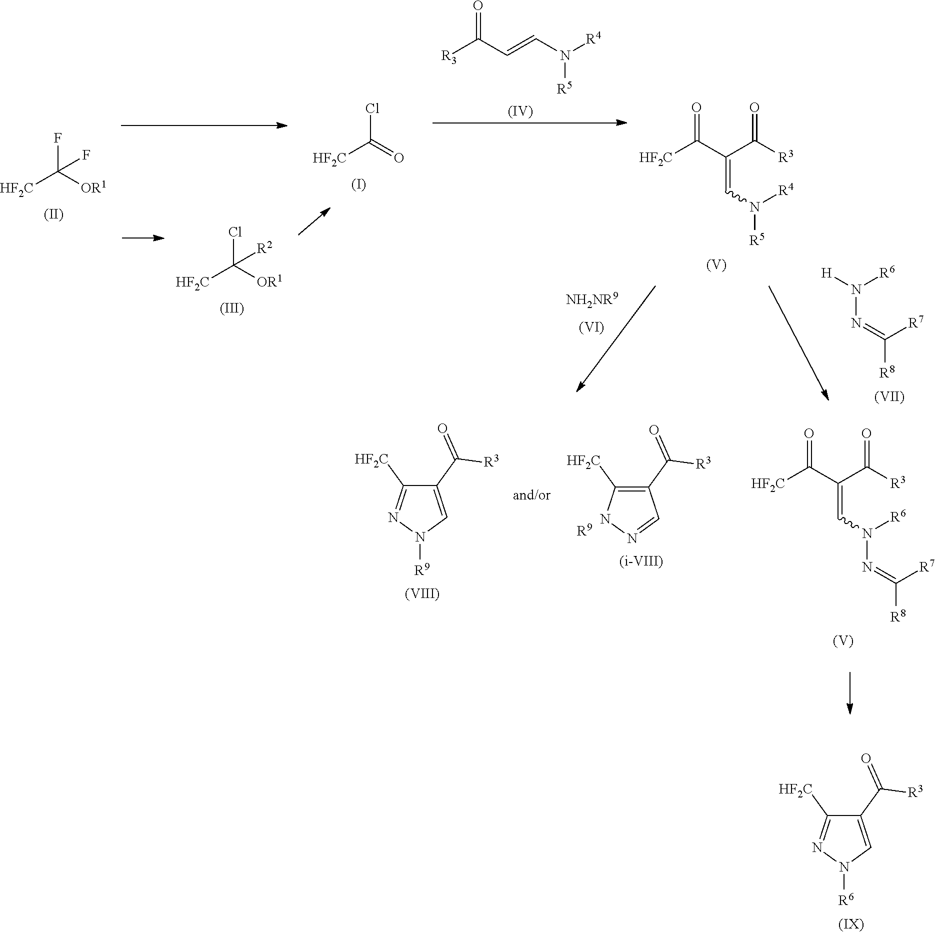

In the above reaction scheme, R¹ and R² are defined as before; R³ is selected from the group consisting of R¹⁰, OR¹¹ and R¹²; R⁴ and R⁵ each independently are selected from the group consisting of H, C₁-C₁₂-alkyl, C₂-C₆ alkenyl, C₃-C₁₀-cycloalkyl, C₂₋₁₂ alkynyl, aryl, heteroaryl, aralkyl or alkylaryl group, each of which is optionally substituted, or R⁴ and R⁵ together with the nitrogen atom to which they are bound form an optionally substituted 5- to 10-membered heterocyclic radical which, in addition to the nitrogen atom, may contain a further 1, 2 or 3 heteroatoms selected from the group consisting of 0, N and S as ring members, wherein R⁴ and R⁵ preferably are methyl; R¹⁰ is selected from the group consisting of C₁-C₁₂-alkyl, C₂-C₆ alkenyl, C₃-C₁₀-cycloalkyl, aryl and heteroaryl, wherein C₁ to C₄ alkyl is preferred and methyl is most preferred; R¹¹ is selected from the group consisting of C₁-C₁₂-alkyl, C₂-C₆ alkenyl, C₃-C₁₀-cycloalkyl, aryl and heteroaryl; R¹² is the group CF₃, CCl₃ or CBr₃, wherein CCl₃ is preferred; R⁹ is selected from the group consisting of H, C₁-C₁₂-alkyl, C₂-C₆ alkenyl, C₃-C₁₀-cycloalkyl, C₂₋₁₂ alkynyl, aryl, heteroaryl and alkyl substituted by aryl; R⁶ is selected from the group consisting of C₁-C₁₂-alkyl, C₂-C₆ alkenyl, C₃-C₁₀-cycloalkyl, C₂₋₁₂ alkynyl, aryl, heteroaryl and alkyl substituted by aryl; R⁷ and R⁸ each independently are selected from the group consisting of H, C₁-C₁₂-alkyl, C₂-C₆ alkenyl, C₃-C₁₀-cycloalkyl, C₂₋₁₂ alkynyl, aryl, heteroaryl, aralkyl or alkylaryl group, each of which is optionally substituted, or R⁵ and R⁶ together with the nitrogen atom to which they are bound form an optionally substituted 5- to 10-membered heterocyclic radical which, in addition to the nitrogen atom, may contain a further 1, 2 or 3 heteroatoms selected from the group consisting of O, N and S as ring members, wherein in one preferred aspect, R⁵ and R⁶ are methyl or wherein in a further preferred aspect, R⁵ is H and R⁶ is phenyl.

When (VIII) is obtained, (i-VIII), which is the isomer of (VIII), can also be obtained.

When R³ in (VIII) or (IX) is R¹², the compound of formula (IX) and/or (VIII) can be converted into a compound of formula (X) by reaction with a base, such as aqueous alkali or earth alkali hydroxide, or formula (IX) and/or (VIII) can be converted into carboxamides of formula (XI) by reaction with a compound of formula (XII) NHR¹³Q, wherein R¹³ is selected from the group consisting of H, C₁-C₁₂-alkyl, C₂-C₆ alkenyl or C₃-C₈-cycloalkyl group, wherein H and C₁-C₄-alkyl are preferred, and wherein Q is an optionally substituted aryl or heteroaryl group. The compound of formula (XI) can be, for example, Sedaxane, Fluopyram, Benzovindiflupyr, Bixafen, Fluxapyroxad, Isopyrazam, Penflufen and Penthiopyrad. The compound of formula (X) can also be converted into the compound of formula (XI) by first converting the compound of formula (X) in the acid halide according to methods known to the person skilled in the art, and wherein the obtained pyrazoles carboxylic halide is contacted with a compound of formula (XII).

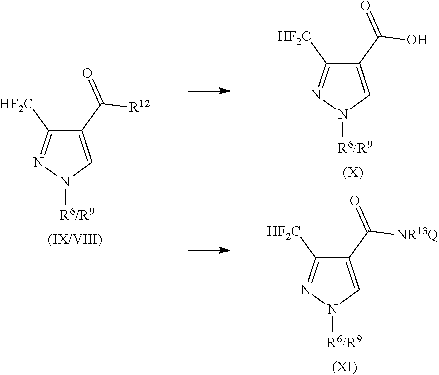

When R³ in (VIII) or (IX) is R¹⁰, the compound of formula (IX) and/or (VIII) can be converted into a compound of formula (X) by reaction with an oxidant, for example oxygen in the presence of a metal catalyst, peracids, halogen such as chlorine, bromine, iodine, oxo acids of halogens and salts thereof, such as hypochlorous acid and salts thereof, hypobromous acid and salts thereof, chlorous acid and salts thereof, iodic acid and salts thereof, periodic acid and salts thereof, peroxides such as hydrogen peroxide. It is preferred that the compound of formula (VIII) or (IX) wherein R³ is R¹⁰ is oxidized with hypochlorite, hypobromite or oxygen in the presence of a metal catalyst.

The processes according to the present invention generally allow for access to DFAC from 1-alkoxy-1,1,2,2-tetrafluoroethane under mild conditions, with high yields. The processes generally are efficient and use less energy by being able to operate at temperatures which are often as low as room temperature. This also often makes it possible to, if desired, perform the process in the liquid phase or in a liquid/solid heterogeneous environment. This can also contribute to easier workup of the reaction, such as liberation of gaseous co-products. It is further believed, without wanting to be bound to a particular theory, that the processes according to the present invention leading to the compound of formula (I) mainly and preferably proceed via the compound of formula (III), even when (III) is not detected or isolated as intermediate, thus providing mainly and preferably for the liberation of an alkyl chloride (such as ethyl chloride) rather than a alkyl fluoride, wherein the alkyl chloride considered to be less harmful and easier to dispose of.

Should the disclosure of any patents, patent applications, and publications which are incorporated herein by reference conflict with the description of the present application to the extent that it may render a term unclear, the present description shall take precedence.

The following examples are intended to further explain the invention without limiting it.

The starting materials are available from commercial sources, or can be made according to processes known to the person skilled in the art. The manufacture of 1-ethoxy-1,1,2,2-tetrafluoroethane is known, for example, from U.S. Pat. No. 2,409,274.

### EXAMPLE 1 DFAC FROM 1-ETHOXY-1,1,2,2-TETRAFLUOROETHANE

1-ethoxy-1,1,2,2-tetrafluoroethane was mixed with AlCl₃ (0.2 eq) and SiCl₄ (0.4 eq) and stirred at room temperature for one hour. A GC measurement showed complete conversion of the 1,1-dichloro-1-ethoxy-2,2-difluoroethane, wherein more than 99% DFAC was detected by GC-MS and NMR and compared with analytical standard.

### EXAMPLE 2.1 1,1-DICHLORO-1-ETHOXY-2,2-DIFLUOROETHANE FROM 1-ETHOXY-1,1,2,2-TETRAFLUOROETHANE IN ETHYL ACETATE

1-ethoxy-1,1,2,2-tetrafluoroethane was mixed with TiCl₄ (1 eq) in ethyl acetate and heated to 80° C. for 18 hours. The reaction mixture was submitted to GC measurement and was found to contain 91% 1,1-dichloro-1-ethoxy-2,2-difluoroethane.

### EXAMPLE 2.2 1,1-DICHLORO-1-ETHOXY-2,2-DIFLUOROETHANE AND DFAC FROM 1-ETHOXY-1,1,2,2-TETRAFLUOROETHANE IN ACETONITRILE

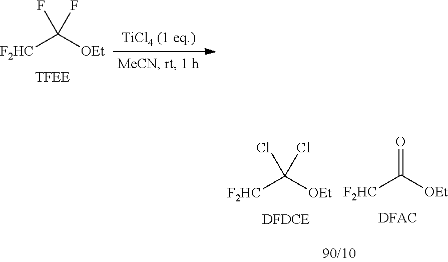 [SMILES: CCOC(F)(F)C(F)F ; CCOC(Cl)(Cl)C(F)F ; CCOC(=O)C(F)F]

A mixture of 1-ethoxy-1,1,2,2-tetrafluorethane (0.5 g, 3.42 mmol, 1.00 eq) and acetonitrile (5.0 mL) was treated with titanium tetrachloride (0.65 g, 3.42 mmol, 1.00 eq) in a sealed vial at 80° C. for five hours. Complete conversion to DFDCE (80%) and DFAC (20%) was observed using ¹H-NMR analysis

### EXAMPLE 3 1,1-DICHLORO-1-ETHOXY-2,2-DIFLUOROETHANE FROM 1-ETHOXY-1,1,2,2-TETRAFLUOROETHANE

1-ethoxy-1,1,2,2-tetrafluoroethane is mixed with TiCl₄ (1 eq) in ethyl acetate and stirred at 23° C. for 18 hours. The reaction mixture is submitted to GC measurement and essentially complete conversion into 1,1-dichloro-1-ethoxy-2,2-difluoroethane is found.

### EXAMPLE 4 DFAC FROM 1-ETHOXY-1,1,2,2-TETRAFLUOROETHANE IN THE PRESENCE OF SICL₄ AND ALCL₃

Aluminium chloride (5.5 g, 41.2 mmol, 0.40 eq) was filled into a steel reactor with Teflon inlet. Then silicon tetrachloride (8.7 g, 51.2 mmol, 0.50 eq) and TFEE (1-ethoxy-1,1,2,2-tetrafluorethane) (15.0 g, 102.7 mmol, 1.00 eq) were added, the reactor was rapidly sealed and the reaction mixture was stirred one hour at room temperature. The pressure was released through a flask cooled to −40° C. Then the reactor was heated to 50° C. and the distillate was collected. After complete transfer 18.2 g of a colourless liquid, containing 71% of DFAC (difluoroacetyl chloride) and 29% of EtCl (ethyl chloride), was obtained.

### EXAMPLE 5 DFAC FROM 1-ETHOXY-1,1,2,2-TETRAFLUOROETHANE WITH ALCL₃ IN TETRACHLOROETHYLENE

Aluminium chloride (0.45 g, 3.38 mmol, 1.00 eq) was filled in a vial under inert atmosphere and then suspended in tetrachloroethylene (1.5 mL). Then 1-ethoxy-1,1,2,2-tetrafluorethane (0.5 g, 3.42 mmol, 1.00 eq) was added, vial sealed and reaction mixture was stirred one hour at 50° C. 1H-NMR showed complete conversion to DFAC.

### EXAMPLE 6 DFAC FROM 1-ETHOXY-1,1,2,2-TETRAFLUOROETHANE VIA DFDCE WITH TICL₄ IN TETRACHLOROETHYLENE

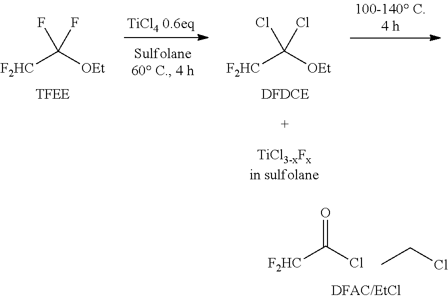

Sulfolane (157 g) was preheated to 50° C. in a flask equipped with a condenser cooled to −20° C., before titanium tetrachloride (32.5 g, 171 mmol, 0.50 eq) was added and the mixture was stirred one hour at 50° C. Then 1-ethoxy-1,1,2,2-tetrafluorethane (50.0 g, 342 mmol, 1.00 eq) was added and the mixture was stirred 4 h at 60° C. Titanium tetrachloride (13.0 g, 68 mmol, 0.20 eq) was added and the temperature was reduced to 50° C. for additional 3.5 h; NMR analysis showed 98.9% conversion. The reaction mixture was cooled down with ice and then stored overnight in the fridge. The next day a Raschig-fitted column equipped with a distillation head and a condenser cooled at −20° C. was installed. The thermal cracking was started at 100° C., until reflux was observed in the column. After four hours of cracking the temperature was increased to 140° C. for 2 h and the distillate was transferred into cooled flasks (−78° C.) in several fractions to give a colorless liquids giving an overall calculated DFAC yield (combined fractions) of 81%.

### EXAMPLE 7 1,1-DICHLORO-1-ETHOXY-2,2-DIFLUOROETHANE FROM 1-ETHOXY-1,1,2,2-TETRAFLUOROETHANE WITH AL₂O₃ AND SICL₄

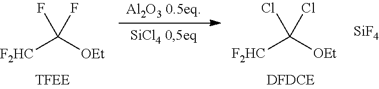

Alumina (36.3 g, 356 mmol, 0.52 eq) was dried in a three-necked-flask. Then the flask was equipped with a condenser cooled to −20° C. and a temperature probe. Silicon tetrachloride (60.5 g, 356 mmol, 0.52 eq) was introduced in one portion and stirring was started. Then 1-ethoxy-1,1,2,2-tetrafluorethane (100.0 g, 685 mmol, 1.00 eq) was added slowly under ice-cooling maintaining the temperature at 10-15° C. The formed gas (silicon tetrafluoride) was allowed to evaporate and was scrubbed with potassium hydroxide. After two hours of stirring at 10° C., the reaction mixture was allowed to warm up to room temperature for 2.5 hours; ¹H-NMR analysis showed complete conversion. The fine suspension was filtered and the filtrate was distilled at 60 mbar and 46° C. to give 78.7 g of 1-ethoxy-1,1-dichloro-2,2-difluoroethane (purity: 99.9% (GC); yield: 64%).

1. A process for the manufacture of a compound of formula (I) or at least one compound of formula (III), or a mixture of the compound of formula (I) and at least one compound of formula (III), wherein a compound of formula (II) is reacted with at least one chloride compound selected from the group consisting of SiCl₄, TiCl₄, ZnCl₂ and AlCl₃ 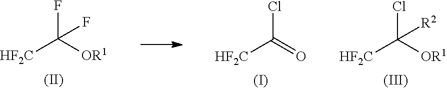
wherein R¹ is selected from the group consisting of C₁-C₁₂-alkyl groups, C₃-C₁₀-cycloalkyl groups, aryl and heteroaryl groups, and wherein R² is F or Cl.

2. A process for the manufacture of a compound of formula (I), wherein at least one of the compounds of formula (III) is reacted with at least one compound selected from the group consisting of chloride compounds SiCl₄, TiCl₄, ZnCl₂, phosphorus pentachloride, thionyl chloride, SbCl₃, SbCl₅, AlCl₃ and metal oxide, 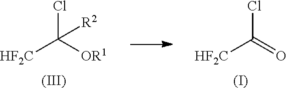
wherein herein R¹ is selected from the group consisting of C₁-C₁₂-alkyl, C₃-C₁₀-cycloalkyl, aryl and heteroaryl, and wherein R² is F or Cl.

3. The process according to claim 2, wherein the at least one compound of formula (III) is manufactured by reacting a compound of formula (II) with at least one compound selected from the group consisting of chloride compounds SiCl₄, TiCl₄, ZnCl₂ and AlCl₃,

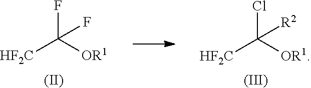

4. The process according to claim 1, wherein R¹ is selected from the group consisting of C₁ to C₄ alkyl groups.

5. The process according to claim 4, wherein R¹ is selected from the group consisting of ethyl and methyl.

6. The process according to claim 1, wherein when the at least one compound of formula (III) is manufactured by reaction of a compound of formula (II), the chloride compound is TiCl₄.

7. The process according to claim 1, wherein when the compound of formula (I) is manufactured by reaction of a compound of formula (II), the chloride compound is SiCl₄, AlCl₃ or a mixture of SiCl₄ and AlCl₃.

8. The process according to claim 7, wherein when the compound of formula (I) is manufactured by reaction of a compound of formula (II), the chloride compound is a mixture of SiCl₄ and AlCl₃.

9. The process according to claim 1, wherein the reaction temperature is from 0° C. to 90° C.

10. The process according to claim 1, wherein the reaction is performed in the absence of additional solvents.

11. The process according to claim 1, wherein the reaction is performed in the presence of at least one solvent.

12. A compound of formula (III) 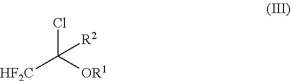
wherein R¹ is selected from the group consisting of C₁-C₁₂-alkyl, C₃-C₁₀-cycloalkyl, aryl and heteroaryl, and wherein R² is F or Cl.

13. The compound according to claim 12, wherein R¹ is selected from the group consisting of C₁ to C₄ alkyl groups.

14. The compound according to claim 13, wherein R¹ is selected from the group consisting of ethyl and methyl.

15. A process for the manufacture of a pharmaceutically or agrochemically active compound, which comprises the process according to claim 1.

16. The process according to claim 3, wherein when the at least one compound of formula (III) is manufactured by reaction of a compound of formula (II), the chloride compound is TiCl₄.

17. The process according to claim 9, wherein the reaction temperature is from 10° C. to less than 50° C.

18. The process according to claim 2, wherein R¹ is selected from the group consisting of C₁ to C₄ alkyl groups.

19. The process according to claim 18, wherein R¹ is selected from the group consisting of ethyl and methyl.
